(19) United States
(12) Patent Application Publication
(10) Pub. No.: US 2005/0260738 A1
(43) Pub. Date: Nov. 24, 2005

(54) Optimized fermentation process of mycelia on the solid medium of starch-processing waste

(76) Inventors: Seokhwan Hwang, Pohang-shi (KR); Seungyong Lee, Seogwipo-shi (KR); Hyokwan Bae, Ulsna-shi (KR)

Correspondence Address: LUCAS & MERCANTI, LLP, 475 PARK AVENUE SOUTH, 15TH FLOOR, NEW YORK, NY, 10016, US

(21) Appl. No.: 11/133,562

(22) Filed: May 20, 2005

(30) Foreign Application Priority Data
May 24, 2004 (KR) 10-2004-0036875

(51) Int. Cl. C12N 1/14; A01G 1/04

(52) U.S. Cl. 435/254.1; 47/1.1

(57) ABSTRACT

The present invention relates to an optimized fermentation process of mycelia on the solid medium of starch-processing waste, more precisely, an optimized fermentation process for mass-production of in variety of highly valuable mycelia on the solid medium prepared only from starch-processing waste, a by-product of the production of starch, without any additives. The optimized fermentation process of mycelia using the solid medium of starch-processing waste excluding any other additives provided by the present invention has many advantages; it is effective for the treatment of starch-processing waste, it is effective for the increase of productivity of mycelia and for shortening of culture time because it enables the rapid growth of mycelia, and it is effective for the mass-production of in variety of highly valuable mycelia which is in great demand.

### TECHNICAL FIELD

The present invention relates to an optimized fermentation process of mycelia on the solid medium of starch-processing waste, more precisely, an optimized fermentation process for mass-production of in variety of highly valuable mycelia on the solid medium prepared only from starch-processing waste, a by-product of the production of starch, without any additives.

### BACKGROUND ART

Starch-processing waste, a by-product of the production of starch, increased upto 1.5 million tons in 2002, and is still increasing every year with the increase of the production of corn and potatoes like sweet potato and potato. The treatment of starch-processing waste largely depends on landfill (70%) and ocean disposal (25%) by a waste dealer, which costs more than 50 billions won annually, being a huge burden on companies and the nation. Therefore, it is inevitable to provide a method and/or techniques for the treatment of starch-processing waste to reduce environmental contamination and to promote re-cycling of waste.

Starch-processing waste does not include any hazardous substance and is in fact mostly composed of carbohydrates and water. Thus, the simple dump-out of such waste is inefficient, and improved treatment hiring pro-environmental recycling techniques is required. As a part of such endeavors, recycling processes of the waste, that is the uses for the culture of edible mushrooms, the production of compost/liquid fertilizer, the extraction of biologically active compounds and the production of active carbon, etc, have been developed.

In particular, mushrooms have been in great demand as food or medicinal additives since they were proved to contain biologically active compounds effective for the treatment of cancer and for the improvement of immunity. Thus, mass-production of mycelia, instead of fruitbody, which seems to be better candidate for the mass-production, is required. The possibility of using starch-processing waste as the medium for the culture of mycelia has been studied in many researches because it might enable mass-production of mycelia with low price and effective recycling of the waste. However, persuasive and systematic optimized process using starch-process waste only has not been proposed, yet, and it is still far from industrial uses of it.

The growth of mycelia is highly sensitive to the concentration and the pH of substrate and temperature as well. In addition, the concentrations of nutritions also affect the growth. However, no guideline or techniques for the culture of highly valuable mycelia on the solid medium of starch-processing waste, a by-product of the production of starch, without any other factor has been reported, letting the efficiency of using starch-processing waste in doubt.

### DISCLOSURE

### Technical Problem

It is an object of the present invention to provide an optimized fermentation process of mycelia by determining the optimum concentration, pH and temperature for the growth of mycelia on the solid medium prepared from starch-processing waste only without any other additional nutrients for the mass-production of mycelia which is effective for the treatment of cancer and the increase of immunity.

In the present invention, experiments were designed to determine optimum conditions for the production with variants of substrate concentration, pH and temperature, based on response surface methodology, the statistical/mathematical optimization technique. And the present inventors completed this invention by determining optimum growth conditions for mycelia on the medium by measuring longitudinal growth rate of mycelia as the growth rate.

### Technical Solution

In order to achieve the above object, the present invention provides optimum conditions for the maximum growth of mycelia determined by using response surface methodology, the statistical/mathematical optimization technique.

The present invention provides optimum culture conditions for mycelia on the solid medium prepared from only starch-processing waste without any nutritional additives.

### DESCRIPTION OF DRAWINGS

FIG. 1 is a set of graphs showing the possibility of mycelia growth on the solid medium of starch-processing waste and optimum concentration for the growth. FIG. 1a is a graph showing the growth rate of Agaricus blazei Murill mycelia according to the concentrations, FIG. 1b is a graph showing the growth rate of Cordyceps militaris mycelia according to the concentrations, FIG. 1c is a graph showing the growth rate of Ganoderma lucidum mycelia according to the concentrations, FIG. 1d is a graph showing the growth rate of Lentinus edodes mycelia according to the concentrations, and FIG. 1e is a graph showing the growth rate of Phellinus linteus mycelia according to the concentrations,

FIG. 2 is a graph showing CCD (Central Composite Design) used in the present invention,

FIG. 3 is a set of graphs showing the contour lines and the three dimensional schemes of response surface indicating the optimum conditions, FIG. 3a shows those of Agaricus blazei Murill mycelia, FIG. 3b shows those of Cordyceps militaris mycelia, FIG. 3c shows those of Ganoderma lucidum mycelia, FIG. 3d shows those of Lentinus edodes mycelia, and FIG. 3e shows those of Phellinus linteus mycelia.

### BEST MODE

Hereinafter, the present invention is described in detail.

The present invention provides optimum conditions for the maximum growth of mycelia determined by using response surface methodology, the statistical/mathematical optimization technique.

The present invention provides optimum culture conditions for mycelia on the solid medium prepared from only starch-processing waste without any nutritional additives.

In the present invention, optimum culture conditions of mycelia on the solid medium of starch-processing waste were obtained. The optimum concentration, pH and temperature for the culture of agaricus (Agaricus blazei Murill) were 40-50 (g/L), 5.5-6.0 and 26±3° C., respectively. For the maximum growth of vegetable worms (Cordyceps militaris), the optimum concentration, pH and temperature were each 20-30 (g/L), 5.3-5.9 and 24±3° C. For the culture of Ganoderma lucidum, the optimum concentration, pH and temperature were 40-50 (g/L), 5.0-5.4 and 30±3° C., respectively. And for the maximum growth of Lentinus edodes, the optimum concentration, pH and temperature were determined respectively to be 35-45 (g/L), 5.0-5.5 and 25±3° C. For the culture of Phellinus linteus, the optimum concentration, pH and temperature were estimated as 20-30 (g/L), 5.5-6.0 and 30±3° C., respectively.

The optimum culture conditions to obtain the best yield of mycelia of Agaricus blazei Murill, Cordyceps militaris, Ganoderma lucidum, Lentinus edodes and Phellinus linteus on the solid medium of starch-processing waste without any additional nutrients were determined by response surface methodology, the statistical/mathematical optimization technique. Longitudinal growth rate was measured to calculate the growth of mycelia, and different central points were applied to 5 different mycelia. Experiments were carried out by central composite design (CCD) in variants ranges of 20 g/L of concentration, pH 2 and 20° C. of temperature. Based on the results obtained from the experiments, the primary and the secondary models and a modified model were used to illustrate response surface, and optimum culture conditions for 5 species of mycelia were estimated.

So, the method of the present invention provides an optimized fermentation process of mycelia on the medium of sterilized starch-processing waste without any other additives, which is very effective for the treatment of starch-processing waste and the increase of mycelia productivity as well as shortening of culture time, owing to the rapid growth of mycelia on the medium.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Strains and Maintenance

Fungal strains used in the present invention, as shown in Table 1, were obtained from the Korean Culture Center of Microorganisms (KCCM) and the Korean Collection for Type Cultures (KCTC) of Korea Research Institute of Bioscience and Biotechnology (KRIBB). The fungal strains were sub-cultured on PDA (Potato Dextrose Agar) medium in Petri dishes in a 25° C. incubator. In order to maintain the strains in physiologically active exponential phase, the strains were continuously sub-cultured before mycelia would cover the whole medium in Petri dish.

TABLE 1
Strain distributors

| | | | Strain |
|---|---|---|---|
| No | Name | Distributor | Number |
| 1 | Agaricus blazei | Korean Culture Center of | KCCM |
| | Murill | Microorganisms (KCCM) | 60257 |
| 2 | Cordyceps | Korean Collection for Type | KCTC |
| | militaris | Cultures (KCTC) | 6472 |
| 3 | Ganoderma lucidum | Korean Collection for Type | KCTC |
| | | Cultures (KCTC) | 6283 |
| 4 | Lentinus edodes | Korean Collection for Type | KCTC |
| | | Cultures (KCTC) | 6735 |
| 5 | Phellinus linteus | Korean Collection for Type | KCTC |
| | | Cultures (KCTC) | 6719 |

### EXAMPLE 2

### Preparation of Sterilized Medium and Inoculation

Starch-processing waste was just dough before the pre-treatment. The dough was dried at 60° C. for 24 hours and pulverized to prepare media of wanted concentrations. The pulverized starch-processing waste was sterilized by autoclaving at 121° C. together with 1.5% agar, which was inoculated into Petri dishes in a germ-free chamber and then was solidified at room temperature. The most active region of each strain, which was kept in exponential phase in PDA medium, was cut by a 5 mm circular blade, and the resultant circular section was transferred onto the medium of starch-processing waste, which was then tightly sealed not to be contaminated.

### EXAMPLE 3

### Application Test of the Strains

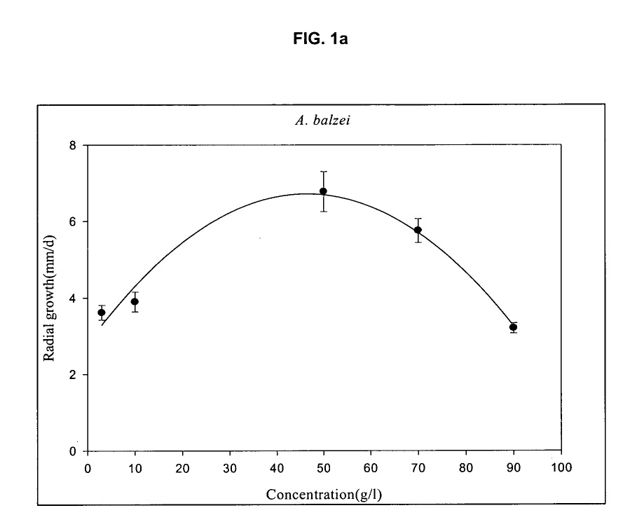

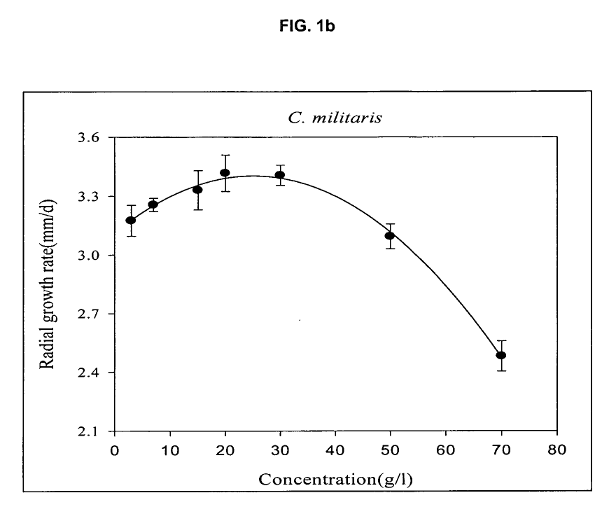

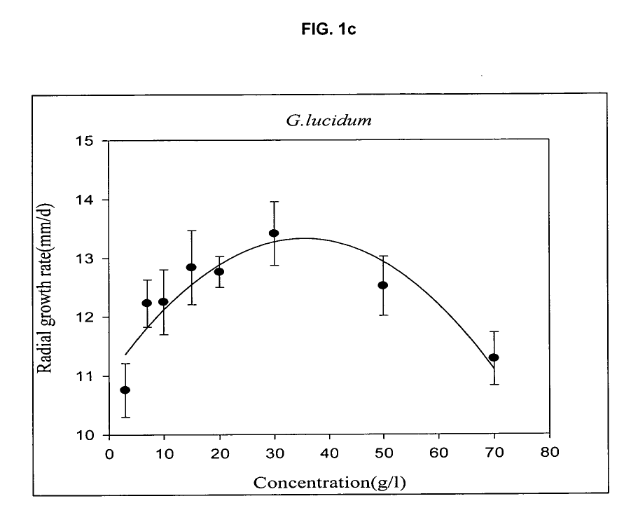

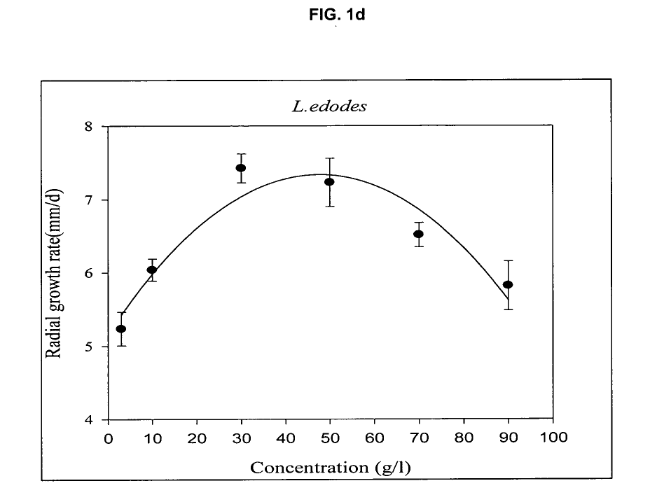

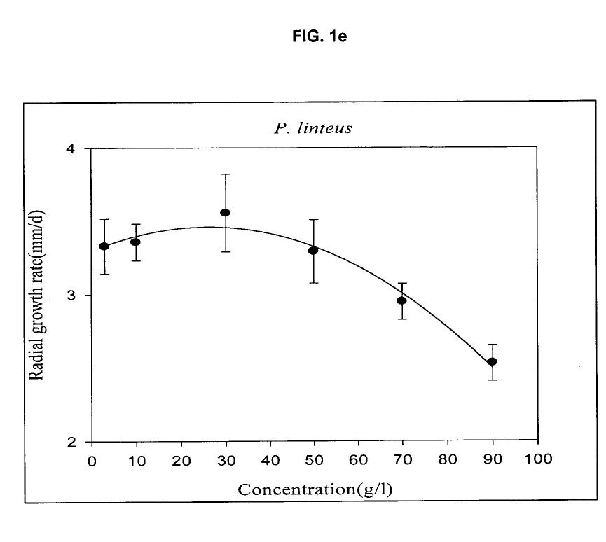

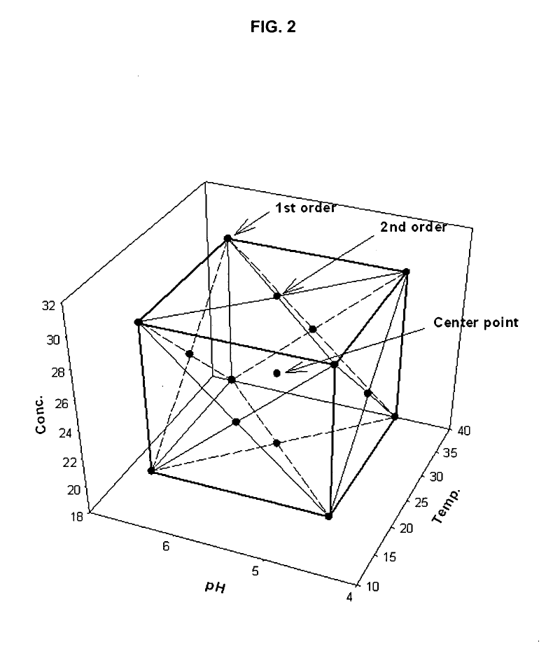

Experiments were designed to confirm the possibility of growth of mycelia on starch-processing waste at 25° C. with optimum pH, proposed by earlier reports. Longitudinal growth rate was measured at different starch-processing waste concentrations of 3, 10, 30, 50, 70 and 90 g/L. Based on the growth rate at each concentration, mathematical polynomial expression calculating the growth rate of mycelia as seen in FIG. 1 was applied to determine optimum concentration, which was then used as a center point of central composite design.

TABLE 2
Optimum substrate concentration and maximum specific
growth rate of mycelia

| | Optimum substrate | Maximum specific |
|---|---|---|
| Name | concentration (g/L) | growth rate Kr(mm/d) |
| Agaricus blazei | 46.4 | 6.7 |
| murrill | | |
| Cordyceps | 25.1 | 3.4 |
| militaris | | |
| Ganoderma | 33.2 | 13.2 |
| lucidum | | |
| Lentinus edodes | 42.6 | 7.5 |
| Phellinus | 26.2 | 3.5 |
| linteus | | |

### EXAMPLE 4

### Design of Experiments and Optimum Conditions

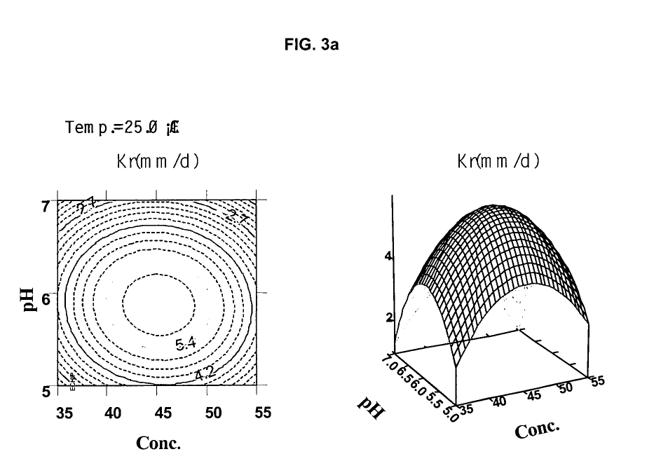

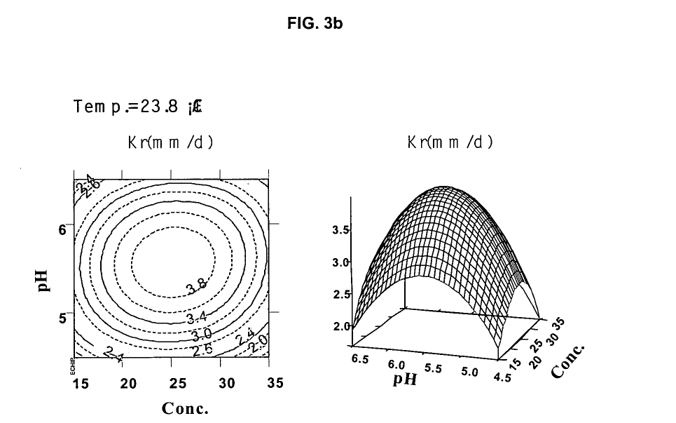

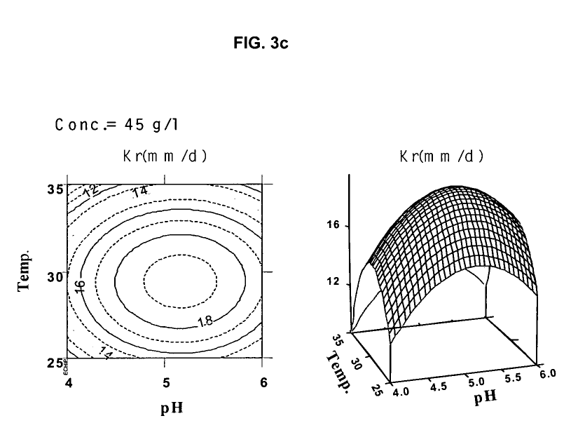

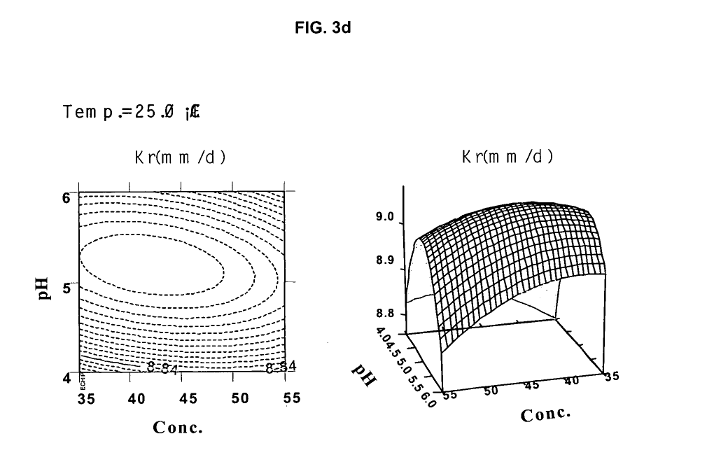

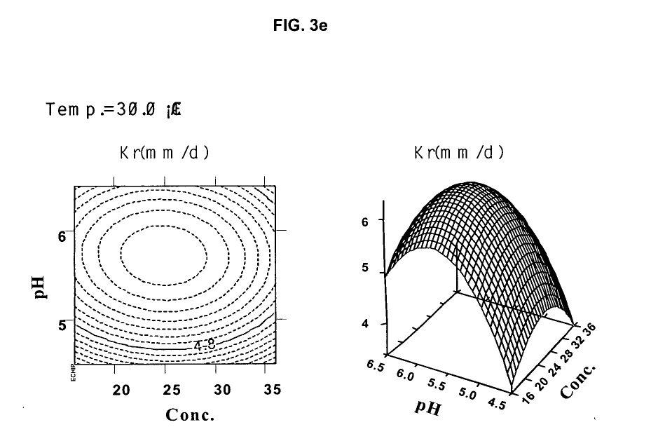

Considering the variants affecting keenly the growth of 5 species of mycelia, substrate concentration, pH and temperature, central composite design was established in the range shown in Table 3 by the procedure shown in Table 4, in order to measure longitudinal growth rate. And the results were analyzed by the secondary or a modified model to determine optimum conditions for the growth of mycelia as shown in FIG. 3. The optimum conditions for the growth of mycelia are shown in Table 5.

TABLE 3
Experimental ranges for the strains

| | Experimental ranges | | |
|---|---|---|---|
| Mycelia | Concentration (g/L) | pH | Temperature (° C.) |
| Agaricus | 35˜55 | 3˜5 | 20˜30 |
| blazei | | | |
| murrill | | | |
| Cordyceps | 15˜35 | 4.5˜6.5 | 20˜30 |
| militaris | | | |
| Ganoderma | 25˜45 | 4˜6 | 25˜35 |
| lucidum | | | |
| Lentinus | 35˜55 | 4˜6 | 30˜30 |
| edodes | | | |
| Phellinus | 16˜36 | 4.5˜6.5 | 25˜35 |
| linteus | | | |

TABLE 4
Examples of experimental design. Experimental design
for Agaricus blazei murrill mycelia and results thereof.

| | | | | | Longitudinal |
|---|---|---|---|---|---|
| Experiment | | Concentration | | Temperature | growth |
| No* | Order | (g/l) | pH | (° C.) | rate (mm/d) |
| 1 | 1st | 35 | 5 | 20 | 1.36 |
| 2 | | 55 | 5 | 20 | 2.45 |
| 3 | | 35 | 7 | 20 | 1.60 |
| 4 | | 55 | 7 | 20 | 1.59 |
| 5 | | 35 | 5 | 30 | 4.58 |
| 6 | | 55 | 5 | 30 | 4.47 |
| 7 | | 35 | 7 | 30 | 3.23 |
| 8 | | 55 | 7 | 30 | 2.57 |
| 9 | | 45 | 6 | 25 | 5.84 |
| 10 | 2nd | 35 | 6 | 25 | 3.50 |
| 11 | | 55 | 6 | 25 | 4.16 |
| 12 | | 45 | 5 | 25 | 4.29 |
| 13 | | 45 | 7 | 25 | 2.60 |
| 14 | | 45 | 6 | 20 | 1.19 |
| 15 | | 45 | 6 | 30 | 4.07 |

※ The results of each experiment No* were obtained through three repeated experiments.

TABLE 5
Optimum conditions of the strains

| | | | | | Expected |
|---|---|---|---|---|---|
| | | Optimum Condition | | | maximum |
| | | Concen- | | Tempera- | specific |
| | | tration | | ture | growth |
| Mycelia | Model | (g/L) | pH | (° C.) | rate (mm/d) |
| Agaricus | Improved | 45.2 | 5.88 | 26 | 5.96 |
| blazei | Model | | | | |
| murrill | | | | | |
| Cordyceps | Improved | 25.1 | 5.58 | 23.8 | 4.10 |
| militaris | Model | | | | |
| Ganoderma | Secondary | 45 | 5.17 | 30 | 18.91 |
| lucidum | Model | | | | |
| Lentinus | Improved | 41.25 | 5.25 | 25 | 9.08 |
| edodes | Model | | | | |
| Phellinus | Improved | 24.72 | 5.71 | 30 | 6.36 |
| linteus | model | | | | |

### INDUSTRIAL APPLICABILITY

As explained hereinbefore, the optimum fermentation process of mycelia on the solid medium of starch-processing waste of the present invention provides the maximum production of mycelia using only starch-processing waste without any additives, which is thus effective not only for the treatment of starch-processing waste but also for the increase of productivity of mycelia and for shortening of culture time as well, owing to the rapid growth of mycelia on the medium. Most of all, the method of the present invention can be effectively used for the mass-production of highly valuable mycelia which is in increasing demand.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An optimized fermentation process of mycelia on the solid medium prepared only from starch-processing waste without any nutritional additives.

2. The optimized fermentation process of mycelia as set forth in claim 1, wherein the mycelia is selected from a group consisting of Agaricus blazei Murill, Cordyceps militaris, Ganoderma lucidum, Lentinus edodes and Phellinus linteus.

3. The optimized fermentation process of mycelia as set forth in either claim 1 or claim 2, wherein the optimum growth conditions for Agaricus blazei Murill mycelia are determined as the concentration of 40-50 (g/L), pH 5.5-6.0 and the temperature of 26±3° C.

4. The optimized fermentation process of mycelia as set forth in either claim 1 or claim 2, wherein the optimum growth conditions for Cordyceps militaris mycelia are determined as the concentration of 20-30 (g/L), pH 5.3-5.9 and the temperature of 24±3° C.

5. The optimized fermentation process of mycelia as set forth in either claim 1 or claim 2, wherein the optimum growth conditions for Ganoderma lucidum mycelia are determined as the concentration of 40-50 (g/L), pH 5.0-5.4 and the temperature of 30±3° C.

6. The optimized fermentation process of mycelia as set forth in either claim 1 or claim 2, wherein the optimum growth conditions for Lentinus edodes mycelia are determined as the concentration of 35-45 (g/L), pH 5.0-5.5 and the temperature of 25±3° C.

7. The optimized fermentation process of mycelia as set forth in either claim 1 or claim 2, wherein the optimum growth conditions for Phellinus linteus mycelia are determined as the concentration of 20-30 (g/L), pH 5.5-6.0 and the temperature of 30±3° C.
